(19) United States
(12) Patent Application Publication
(10) Pub. No.: US 2008/0300456 A1
(43) Pub. Date: Dec. 4, 2008

(54) Video Endoscope

(76) Inventors: Klaus M. Irion, Emmingen-Liptingen (DE); Christian Graf, Emmingen-Liptingen (DE); Peter Schwarz, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 12/130,748

(22) Filed: May 30, 2008

(30) Foreign Application Priority Data
May 31, 2007 (DE) 10 2007 026 234.7

(51) Int. Cl. A61B 1/04; A61B 1/06

(52) U.S. Cl. 600/109; 600/160

(57) ABSTRACT

A video endoscope comprises a disposable working part. The working part comprises a shaft, at the distal end of which a lens and an image sensor are arranged. The working part furthermore comprises an electrical transmission system for transmitting the image information and at least one light guide for transmitting light from proximal to distal. An operating part is furthermore provided. A proximal end of the working part can be releasably connected to a distal end of the operating part via an interface. Furthermore, a sterile gathered-together hose is arranged on the working part, and the operating part can be enveloped by the hose.

### BACKGROUND OF THE INVENTION

The invention relates to a video endoscope comprising a disposable working part, wherein the working part comprises a shaft, at the distal end of which a lens and an image sensor are arranged, and wherein the working part furthermore has an electrical transmission system for transmitting the image information and at least one light guide for transmitting light from proximal to distal, and comprising an operating part, wherein a proximal end of the working part can be releasably connected to a distal end of the operating part via an interface.

Video endoscopes are used for example in minimally invasive interventions in arthroscopy, laparoscopy and thoracoscopy, for inguinal hernias and in operations on the joints and spinal column. Endoscopes thereby assist the operating surgeon in carrying out the operation, by providing the largest possible range of view of the operating site.

A first part of endoscopes of this type is an imaging system. The imaging system serves for receiving observation light from the observation space or operation space and for transmitting image information from distal to proximal.

The imaging system can conventionally comprise an optical image transmission system having a lens in the distal end of the shaft, a proximally adjacent lens system, for example in the form of a rod lens system, or an ordered fibre bundle and a proximal eyepiece, through which the eye can observe an image or to which a camera can be connected.

An electronic imaging system comprises, in the distal end of the shaft, an imaging optic and an image recording chip, which converts the light signals into electrical signals that are transmitted towards the proximal end via electrical lines. At the proximal end there is a camera module present, which is usually additionally formed as an operating part and forwards the electronic image information to an image processing unit.

A second part of endoscopes of this type is an illumination system. The illumination system serves for transmitting light from proximal to distal in order to illuminate the observation space or examination space with light.

After a surgical intervention has been carried out, a video endoscope of this type has to be cleaned and sterilized by means of an autoclaving process. In practical applications of such video endoscopes it has been found that in particular optical and electronic components of the video endoscopes can be impaired during sterilization or autoclaving processes, which are carried out in the temperature range of 130 to 140° C.

The temperature fluctuations that take place during the autoclaving processes cause considerable mechanical tensile stresses which can lead to fractures of connection locations or at a lens. Furthermore, the highly sensitive electronic parts of the video endoscope, e.g. image sensors with colour filter, can also be impaired as a result. Consequently, the abovementioned processes shorten the lifetime of the endoscope. Moreover, the sterilization or autoclaving processes are time- and cost-intensive.

Accordingly, solutions are sought in which the costly sterilization or autoclaving processes can be dispensed with.

A video endoscope having the structural features mentioned above is known e.g. from JP 06254049 A.

Although providing a disposable working part partially solves the problem of sterilizing the video endoscope in that a working part sterilized beforehand can be connected as a single-use component to an operating part, the problem still exists that the operating part can be soiled and contaminated by fluids and liquids present in the operating theatre, which may lead e.g. to malfunctions in the operating part. Furthermore, the operating part still has to be sterilized, even though this no longer has to be effected to the same high degree as when the operating part is fixedly connected to the working part that is inserted directly into a patient's body. However, even sterilization methods under milder conditions such as e.g. soaking the operating part in a disinfection liquid can lead over the long term to malfunctions through to the failure of the operating part.

Consequently, it is an object of the invention to further develop a video endoscope of the type mentioned in the introduction to the effect that, in a simple manner, the operating part is protected against contamination in the operating theatre and, at the same time, the operating theatre is protected from possible contamination by the operating part.

### SUMMARY OF THE INVENTION

According to the invention, the object on which the invention is based is achieved by means of a video endoscope of the type described in the introduction wherein a sterile gathered-together hose is arranged on the working part, and wherein the operating part can be enveloped by the hose.

This measure has the advantage that the operating part, which comprises a plurality of sterilization-sensitive elements, is protected against contamination by the hose. The working part is connected to the non-sterilized operating part. The working part is then grasped by the operating surgeon's sterile hand and with the second hand the sterile hose arranged on the working part is pulled over the non-sterilized operating part, such that the hose envelops the operating part. After use, the working part is separated from the operating part and the working part with the hose is disposed of. This enveloping process can also be effected with constrained guidance using a docking device.

In a further embodiment of the invention, the interface is formed in such a way that the image information can be transmitted from the image sensor to the operating part.

This measure has the advantage that the coupling of the elements responsible for the image transmission is ensured by such a configuration of the interface. The image information is transmitted from the sensor to the operating element via the interface by means of the electrical transmission system, such that the image information can be represented as an image on a monitor after image processing. As a result, the operating surgeon can observe the endoscopic operation area on the monitor.

In a further embodiment of the invention, the interface is formed in such a way that light can be transmitted from the operating part to the working part.

This measure has the advantage that light is guided from the operating part to the distal end of the working part, whereby the examination space is illuminated with light.

In a further embodiment of the invention, the working part comprises an outer part formed as a shaft.

This measure has the advantage that the shaft fulfils a protective function since the elements situated in the shaft, such as the sensor, transmission system, etc., are protected against being impaired e.g. by the action of warping forces. This shaft can be formed as a plastic shaft and can be implemented as an injection-moulded part. Consequently, it can be implemented extremely cost-effectively.

In a further embodiment of the invention, the at least one light guide is arranged in the shaft.

This measure has the advantage that the shaft likewise protects the light guide in addition to the function of protecting the sensitive elements arranged in the endoscope. The shaft can be formed from a light-guiding plastic. This contributes to a simple and cost-effective construction of the working part.

In a further embodiment of the invention, the working part comprises an inner part, in which the image sensor and the electrical transmission system are arranged.

This measure has the advantage that damage to the image sensor and the image transmission system is prevented by such a configuration of the working part since the elements are enclosed and thus protected by the inner part.

In a further embodiment of the invention, the outer part can be rotated relative to the inner part.

This measure has the advantage that so-called erecting of the image is ensured by the rotatability of the outer part with the lens relative to the image sensor.

Erecting of the image is understood to mean a specific horizontal orientation of the monitor image generated by the camera module after rotation of the endoscope usually with an asymmetrical viewing direction (e.g. 30°) about the shaft axis. Some operating surgeons want a constant horizontal orientation of the operation image visible on the monitor if the endoscope is rotated from an initial position during an operation. For this purpose, it has become known per se for the image recording unit to be correspondingly rotated, in order thereby to erect the image again.

In a further embodiment of the invention, a mechanism for rotating the outer part is provided.

This measure has the advantage that the erecting of the image can be carried out by the actuation of the mechanism in a simple manner, even with one hand, by the operating surgeon during the surgical intervention.

The mechanism can be driven manually e.g. by means of an actuating lever, or in motor-actuated fashion. For driving of the motor-actuated rotation of the outer part, the working part can comprise e.g. a friction region or a toothing and the operating part can comprise a drive roller or a gearwheel. Both the manually driven and the motor-driven mechanism can be realized in a structurally simple manner.

In a further embodiment of the invention, the image sensor is formed as a CMOS sensor.

This measure has the advantage that image sensors of this type have in practice proved to be particularly advantageous with regard to their resolution. CMOS technology, in comparison with CCD sensors, has a significantly lower energy consumption and permits expedient production in the case of a relatively large production volume since they can be manufactured without retooling on the production lines designed for large numbers and thus cause a low production outlay per chip. Consequently, the disposable working part can be produced particularly cost-effectively.

In a further embodiment of the invention, which can be used as an alternative to the embodiment mentioned previously, the image sensor is formed as a CCD sensor.

This measure has the advantage that the CCD sensors are usually more light-sensitive than usual CMOS sensors.

In a further embodiment of the invention, the electrical transmission system is formed as at least one continuous circuit board.

This measure has the advantage that a continuous circuit board represents a structurally simple and at the same time cost-effective embodiment. Furthermore, no additional cable connections and contact-connections are required with such an embodiment of the transmission system. This contributes to the working part having a simple construction with convenience of assembly.

In a further embodiment of the invention, a proximal end of the circuit board is formed as a plug.

This measure has the advantage that the image information is transmitted from the image sensor, which is arranged in the working part, to the operating part through the plug. Furthermore, such an embodiment of the working part has the advantage that the cost-effective circuit board is formed in multifunctional fashion, such that no additional components need to be used. This contributes to a lower assembly outlay.

In a further embodiment of the invention the image sensor and the circuit board are enclosed by a cylindrical element.

This measure has the advantage that the entire arrangement comprising the image sensor and the circuit board is stabilized or reinforced by the cylindrical element. On the other hand, the cylindrical element also advantageously serves for insulating the conductor wires.

In a further embodiment of the invention, the working part comprises at least one channel.

This measure has the advantage that the at least one channel can serve e.g. for rinsing and cleaning the lens or for inserting a medical instrument. The channel preferably opens issuing laterally from the shaft, to be precise before the coupling region with the operating part.

In a further embodiment of the invention, the proximal end of the working part has a larger diameter than the distal end.

This measure has the advantage that the distal end region of the working part, by means of such a configuration, has enough space for the electrical plug system and the coaxially arranged light coupling.

In a further embodiment of the invention, LEDs are arranged in the working part.

This measure has the advantage that the LEDs, which can be electrically supplied via the plug, couple light into the outer part.

In a further embodiment of the invention, LEDs are arranged in the inner part of the working part.

This measure has the advantage that the LEDs can couple light radially into the outer part, which is deflected towards the distal end there by means of mirror elements.

In a further embodiment of the invention, a light entrance is arranged at the proximal end of the working part.

This measure in turn has the advantage that light can be transmitted from the operating part to the working part via the light entrance, such that the operation region is illuminated.

In a further embodiment of the invention, a light exit is arranged at the distal end of the operating part.

This measure has the advantage that light can be transmitted from the operating part to the working part through the light exit. The light exit of the operating part is configured in such a way that, after the operating part has been connected to the working part, the light exit is in direct contact with the light entrance of the working part. The light exit comprises either LEDs or optical fibres.

In a further embodiment of the invention, a socket for receiving the plug is arranged at the distal end of the operating part.

This measure has the advantage that an electrical coupling between the working part and the operating part is provided in a structurally simple manner.

In a further embodiment of the invention, the operating part comprises operating elements.

This measure has the advantage that the operating surgeon can operate the camera or other switches or valves by simple actuation of the operating elements arranged on the operating part.

In a further embodiment of the invention, LEDs are arranged circumferentially in the distal region of the operating part, the LEDs illuminating the light entrance of the working part.

This measure has the advantage that the light can be coupled in in a manner distributed uniformly circumferentially, and that this is ensured even when there is rotatability in any rotational position.

In a further embodiment of the invention, optical fibres are arranged in a manner distributed circumferentially in the distal region of the operating part, the fibres illuminating the light entrance of the working part.

This measure has the same advantage as mentioned previously, whereby the light is guided to the distal region of the operating part through optical fibres.

In a further embodiment of the invention, LEDs are arranged in a manner distributed circumferentially in the operating part, the LEDs coupling light radially into the working part, which can be deflected towards the distal end by means of mirror elements.

This possibility of coupling in light also permits an effective coupling in of light in any rotational position of the components among one another.

This type of coupling in may also be present in the working part itself if, by way of example, the working part and operating part are not coupled to one another in a rotatable manner and the working part has an inner part and an outer part rotatable with respect thereto.

Further features and advantages will become apparent from the following description and the accompanying drawings.

It goes without saying that the features mentioned above and those yet to be explained below can be used not only in the combination specified but also in other combinations or by themselves, without departing from the scope of the present invention.

### BRIEF DESCRIPTION OF THE DRAWINGS

The invention will be described and explained in more detail below on the basis of selected exemplary embodiments in association with the accompanying drawings. For the sake of better clarity, the hose according to the invention is not illustrated in FIGS. 2 and 8 to 10. In the figures:

FIG. 1 shows a perspective view of a video endoscope according to the invention, whereby a working part and an operating part are separated from one another;

FIG. 2 shows an enlarged perspective view of a proximal end region of the working part;

FIG. 3 shows an enlarged partially sectional view of a distal end region of the working part;

FIG. 4 shows an enlarged perspective view of the operating part;

FIG. 5 shows an illustration comparable to the illustration in FIG. 1, whereby the operating part and the working part are connected;

FIG. 6 shows an illustration comparable to the illustration in FIG. 1, whereby a further exemplary embodiment of the working part is illustrated;

FIG. 7 shows an illustration comparable to the illustration in FIG. 5, whereby the operating part is enveloped by the sterile hose;

FIG. 8 shows a longitudinal section in the coupling region between a working part and an operating part with different embodiments of the coupling in of light, in the upper half via LEDs in the operating part to optical fibres in the working part, in the lower half from optical fibres in the operating part to optical fibres in the working part;

FIG. 9 shows a longitudinal section at a proximal end of a further embodiment of a working part with a light source in the form of LEDs integrated therein; and

FIG. 10 shows a section in the coupling region between a working part and an operating part, whereby light is radiated from the operating part in the radial direction into the working part, in which it is directed towards the distal end by mirror elements.

### DETAILED DESCRIPTION OF PREFERRED EMBODIMENTS

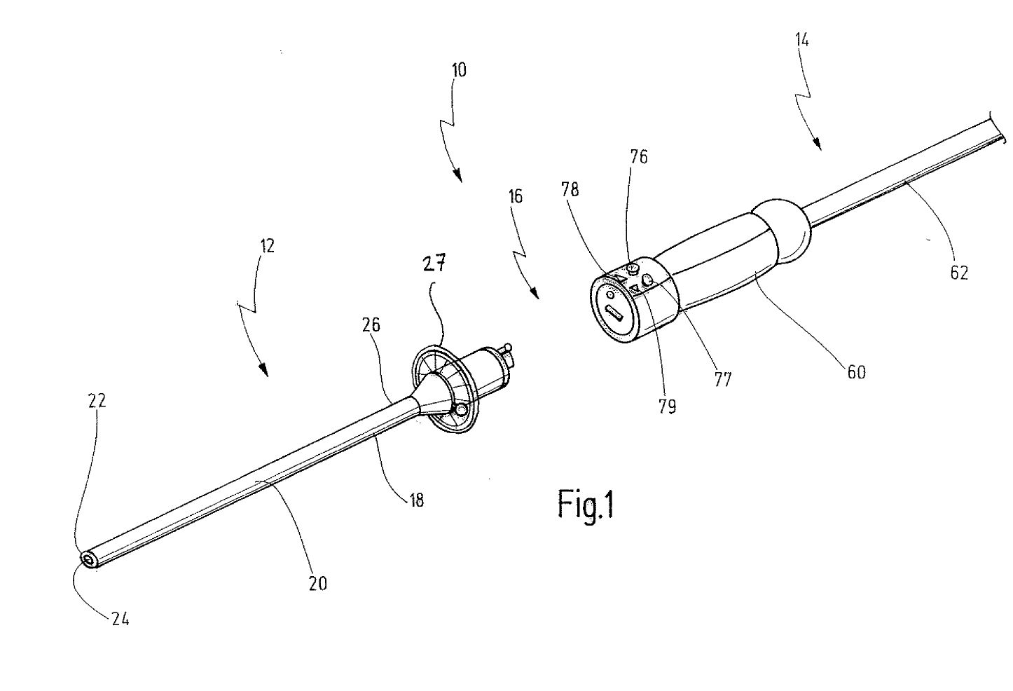

A video endoscope illustrated in FIGS. 1 to 7 is provided in its entirety with the reference numeral 10.

The video endoscope 10 comprises a working part 12 and an operating part 14, which can be releasably connected to one another via an interface 16, as will be explained in more detail later.

The working part 12 is formed as a disposable instrument and the operating part 14 is formed as a reusable instrument.

The working part 12 has an outer part 18, which is formed as a shaft 20 in this exemplary embodiment. The shaft 20 has at least one light guide 26 that guides light from proximal to distal. In this exemplary embodiment, the shaft 20 is formed from a light-guiding plastic.

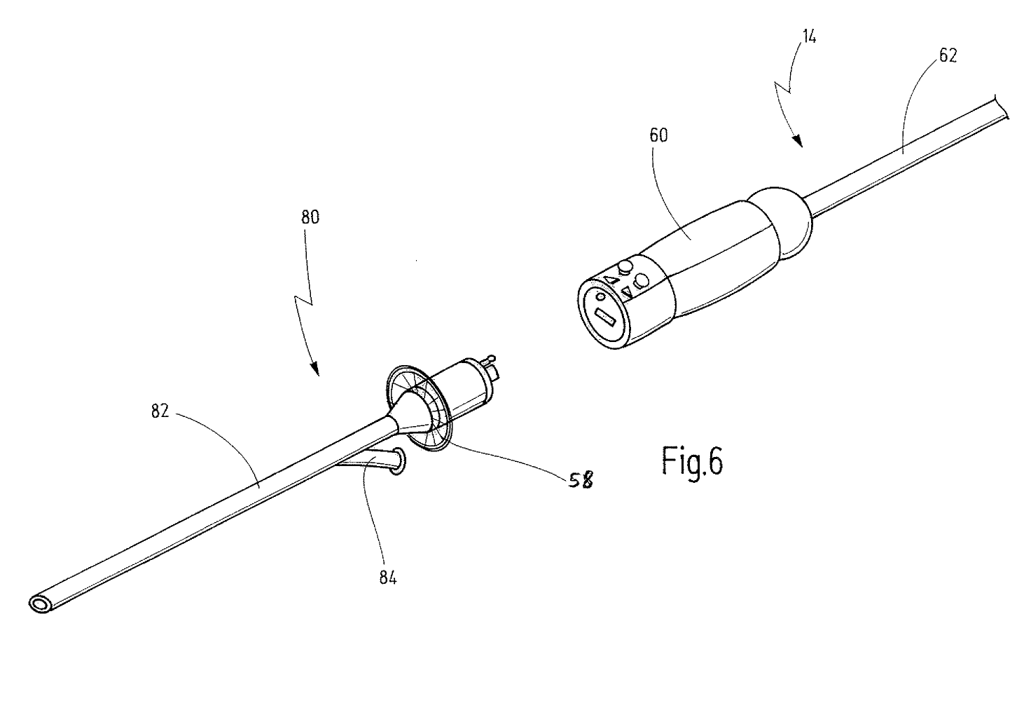

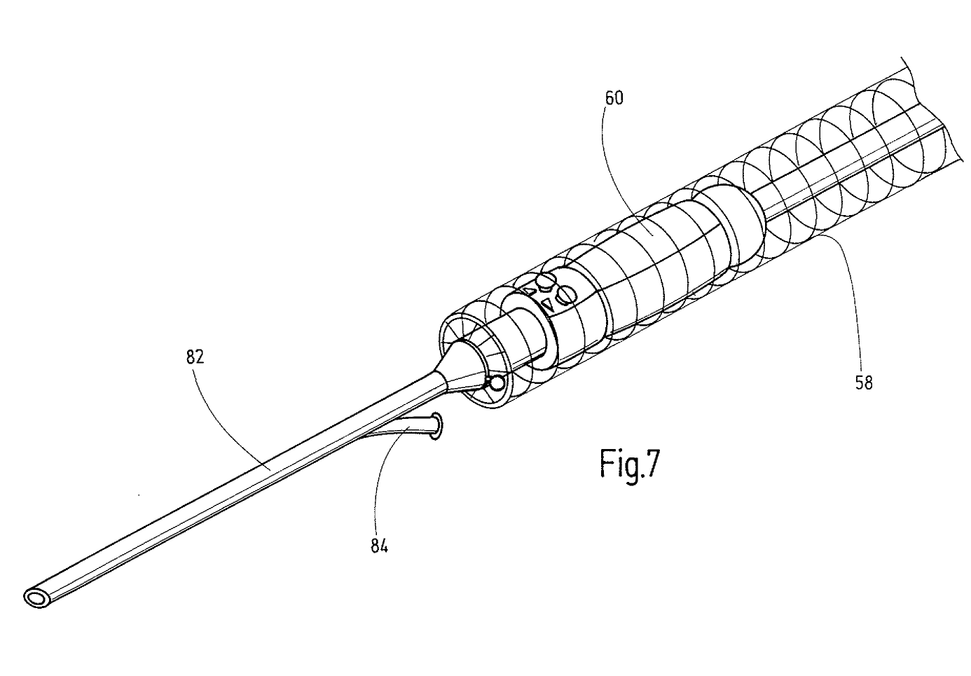

A gathered-together hose 27 is arranged on the proximal end of the working part 12, the precise functioning of such a hose 27 will be explained in more detail below with reference to FIGS. 6 and 7.

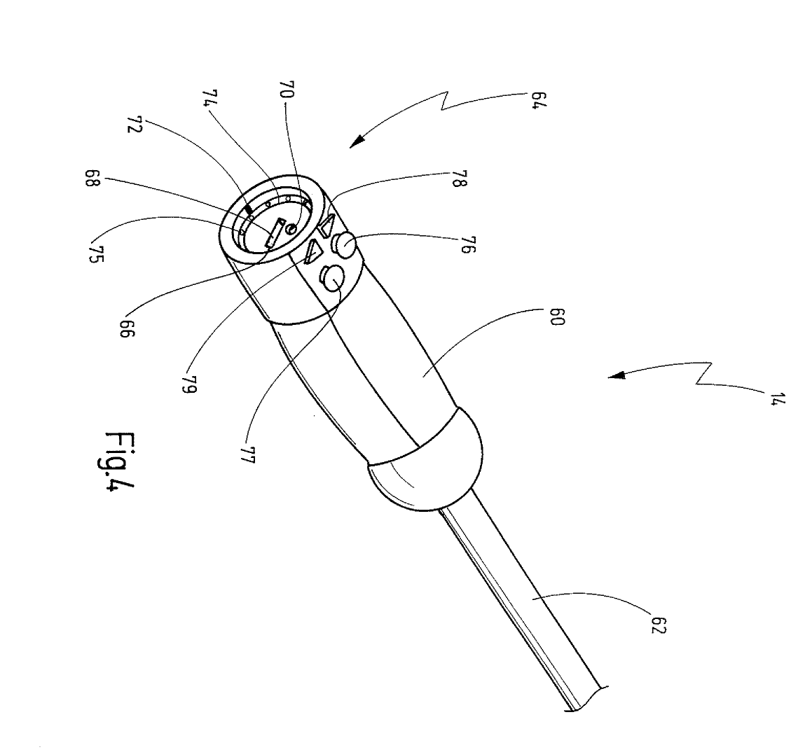

The shaft 20 is closed off hermetically at its distal end 22 with a lens 24, as can be seen in particular from the enlarged illustration in FIG. 3.

The shaft 20 formed from a light-guiding plastic is produced in an injection-moulding process. In this case, part of the lens 24 can concomitantly be injected-moulded at the same time.

The working part 12 furthermore comprises an inner part 30 which can be seen in particular from the illustration in FIG. 3.

Accommodated in the inner part 30 is an image sensor 32, which is formed as a CMOS sensor in this exemplary embodiment. The sensor 32 converts the light signals into electrical signals.

Furthermore, an electrical transmission system 34 is arranged in the inner part 30, which system transmits the image signals from the sensor 32 towards the proximal end. In this exemplary embodiment, the electrical transmission system 34 is formed as a continuous circuit board 36.

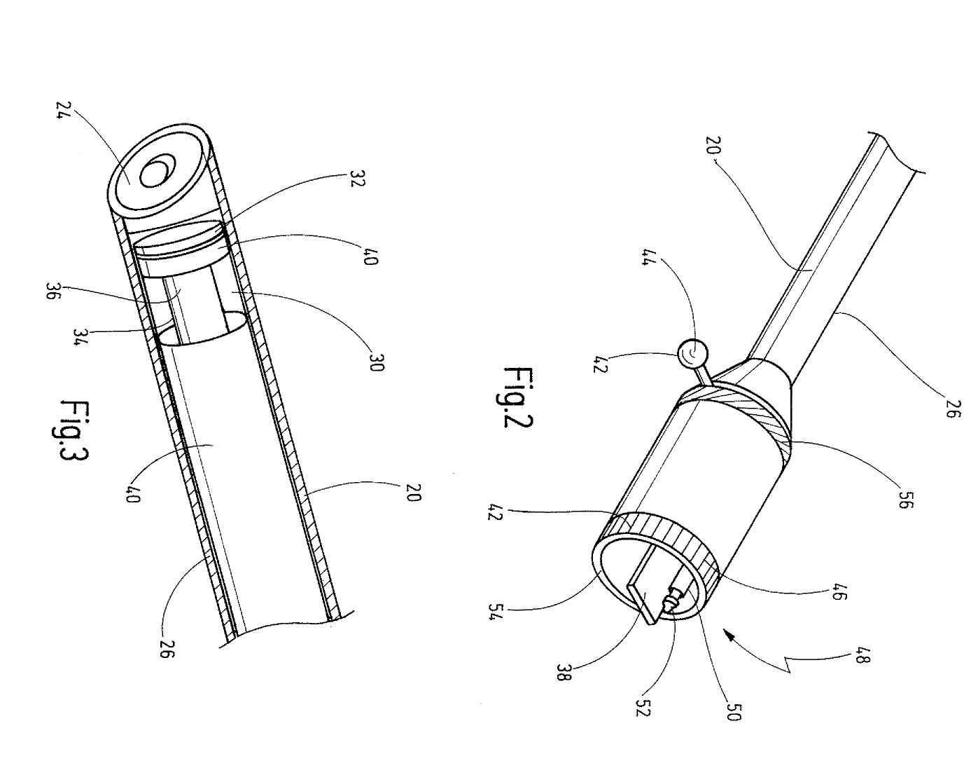

A distal end of the circuit board 36 is formed as a plug 38, which can be seen in particular from the enlarged illustration in FIG. 2. The function of the plug 38 will be described in more detail later.

The image sensor 32 and the electrical transmission system 34 formed as a circuit board 36 are enclosed by a cylindrical element 40. The cylindrical element 40, which is formed from a potting compound, serves for stabilizing the image sensor 32 and the circuit board 36 and for electrical insulation.

The outer part 18 can be rotated relative to the inner part 30. The rotatability of the lens 24 arranged on the distal end of the shaft 20 relative to the image sensor 32 arranged in the inner part 30 enables the image to be erected.

Furthermore, a mechanism 42 for rotating the outer part 18 is provided. The mechanism 42 can be driven manually or in motor-actuated fashion.

An actuating lever 44 is provided for manually driving the mechanism 42, said lever being arranged on the working part 12 at the proximal end. By manually rotating the actuating lever 44, the outer part 18 of the working part 12 is rotated with respect to the inner part 30.

For driving the mechanism 42 in motor-actuated fashion, the working part 12 comprises a toothing or a friction region 46 arranged at the proximal end 48 of the working part 12. By contrast, the operating part 14 comprises at its distal end 64 a gearwheel or a friction wheel 72 which, after the two elements 12 and 14 have been connected, interacts with the toothing or the friction region 46 of the working part 12.

A locking element 50 is arranged at the proximal end 48 of the working part 12, said locking element serving for locking the working part 12 on the operating part 14. In this exemplary embodiment, the locking element 50 is formed as a pin 52.

Furthermore, a ring-shaped light entrance 54 is arranged at the proximal end 48 of the working part 12, by means of which light entrance 54 light is transmitted from the operating part 14 to the working part 12.

In this case, the region 56 represents the region at which the hose 27 (not illustrated here) is connected to the working part 12.

The operating part 14 is formed as a handle 60, as is apparent in particular in FIG. 4. The handle 60 is connected to a cable 62. Sterilization-sensitive, electronic elements, which are not illustrated here, are accommodated in the operating part 14.

At its distal end 64, the operating part 14 comprises a socket 66 comprising a slot 68, into which the plug 38 of the circuit board 36 is inserted. This provides an electrical coupling which enables the image information to be transmitted from the image sensor 32 to the operating part 14. After image processing, the transmitted image information becomes visible to the operating surgeon as an image on a monitor.

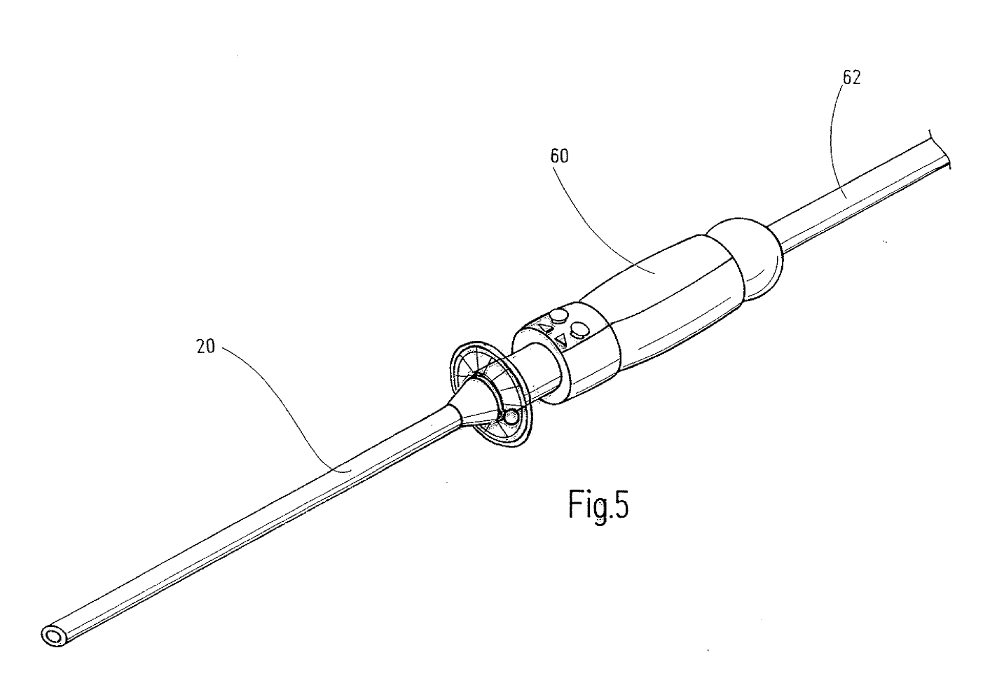

Furthermore, a cut-out 70 is provided at the distal end 64 of the operating part 14, into which cut-out 70 the locking element 50—formed as pin 52—of the working part 12 can be inserted, whereby the working part 12 is connected to the operating part 14. Such a situation is illustrated in FIG. 5.

A light exit 74 is likewise arranged at the distal end 64 of the operating part 14, said light exit 74 being formed in ring-shaped fashion in this exemplary embodiment. In this exemplary embodiment, LEDs 75 are arranged at the light exit 72. The light exit 72 of the operating part 14 is arranged and formed in such a way that, after the working part 12 has been connected to the operating part 14, said light exit 72 is in contact with the light entrance 54 of the working part 12. As a result, light is transmitted from the operating part 14 to the working part 12.

Operating elements 76, 77, 78 and 79 are arranged on the operating part 14 formed as a handle 60, the camera being actuated by means of said operating elements 76, 77, 78 and 79.

FIG. 6 illustrates a further exemplary embodiment of a working part 80, which likewise comprises a shaft 82. The working part 80 comprises the same elements as the working part 12 illustrated in FIGS. 1 to 5. The corresponding elements are provided with the same reference numerals as for the working part 12.

The working part 80 illustrated in FIG. 6 differs from the working part 12 in that it has a laterally emerging instrument channel 84, via which an instrument can be inserted. A hose, for example, can be connected to the end. The instrument channel 84 emerges before the region at which the components are plugged together.

Before a surgical interventions the working part 80 with the sterile hose 58 is removed from a sterile covering (not illustrated) and connected to the non-sterilized operating part 14. The sterile hose 58 fixed to the working part 80 is then pulled over the operating part 14 formed as a handle 60, such that the non-sterilized operating part 14 is enveloped by the sterile hose 58. Such a situation is illustrated in FIG. 7.

This ensures that the sterile working part 80 is not contaminated by the non-sterilized operating part 14. After use, the working part 80 with the sterile hose 58 is separated from the operating part 14 and disposed of.

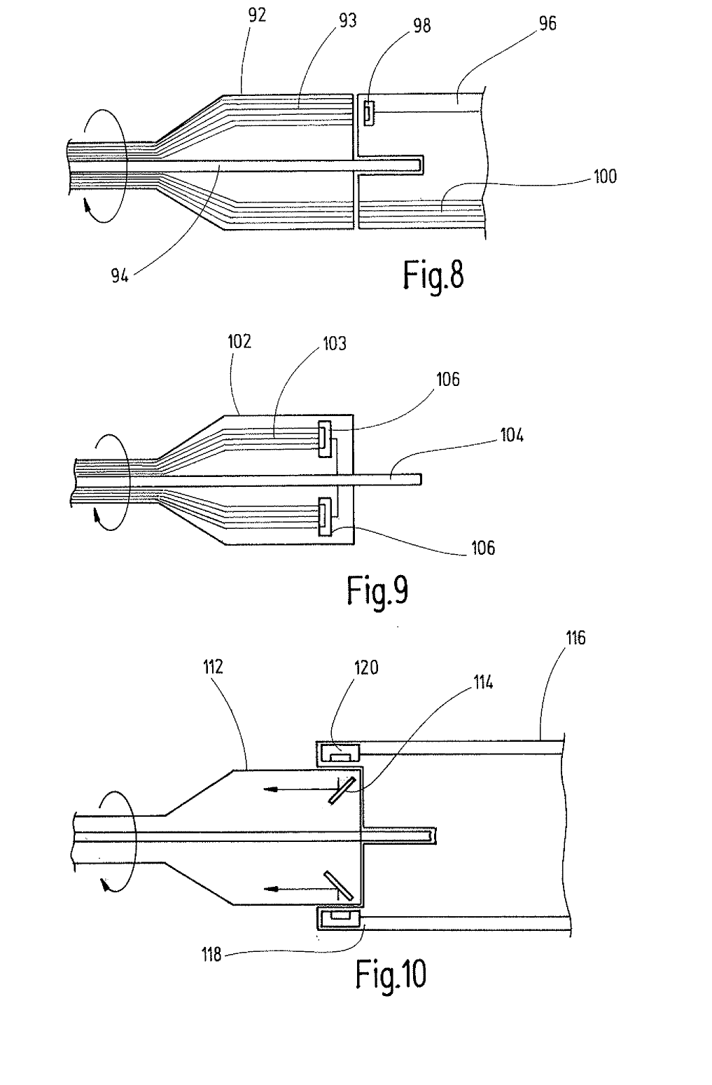

FIG. 8 illustrates a working part 92 coupled to an operating part 96 via a plug 94.

Optical fibres 93 are arranged in the working part 92 in a manner distributed circumferentially, said optical fibres 93 extending from the proximal as far as the distal end of the working part 92. The upper half of FIG. 8 illustrates a variant of coupling light into the working part, namely by means of LEDs 98 arranged correspondingly circumferentially. The lower half illustrates another possibility, namely by means of correspondingly circumferentially distributed optical fibres 100 in the operating part 96.

FIG. 9 illustrates a variant in which optical fibres 103 are arranged in a manner distributed circumferentially in the working part 102, LEDs 106 being arranged at the proximal end of said optical fibres 103. The LEDs 106 are electrically connected to the plug 104 via lines and are supplied with energy via said plug 104 when said working part 102 is coupled to a corresponding operating part. In this case, therefore, the light is generated in the working part 102 itself, namely by means of the LEDs 106, and is then guided to the distal end via the optical fibres 103. The energy is fed in after coupling to the operating part. Consequently, in this case too the working part 102 can be realized as a cost-effective component, that is to say as a disposable part.

FIG. 10 shows a further variant of coupling in light. In this exemplary embodiment, LEDs 120 are arranged in the operating part 116 in a distally projecting circumferential flange 118. These LEDs 120 emit light in a radial direction.

If the operating part 116 is coupled to a working part 112 having mirror elements 114 distributed at the proximal end, the illumination light fed in radially can be deflected and forwarded towards the distal end by said mirror elements. The forwarding can then be effected in various ways, either by means of the material of the working part 112 itself or by means of corresponding optical fibres or the like.

All three variants of FIGS. 8, 9 and 10 permit a relative rotatability between working part and operating part, without the setting having an influence on the illumination.

The variant shown in FIG. 10 is illustrated here between the working part 112 and operating part 116. However, the operating part 116 illustrated in FIG. 10 can also be an outer part of the working part 112, as was described previously in the variant of FIGS. 1 to 5, that is to say that the rotatability is then provided in the working part itself namely between the inner part with the mirror elements and the outer part—surrounding the inner part—with the radially emitting LEDs.

What is claimed is:

1. A video endoscope comprising
a disposable working part having a distal end and a proximal end, wherein said working part comprises
a shaft having a distal end and a proximal end,
a lens and an image sensor, said lens and said image sensor being arranged at said distal end of said shaft,
an electrical transmission system for transmitting image information, and
at least one light guide for transmitting light from proximal to distal,
an operating part having a distal end and a proximal end, wherein said proximal end of said working part can be releasably connected to said distal end of said operating part via an interface, and
a sterile gathered-together hose, said sterile gathered-together hose being arranged on said working part, wherein said operating part can be enveloped by said hose.

2. The video endoscope of claim 1, wherein said interface is formed in such a way that said image information can be transmitted from said image sensor to said operating part.

3. The video endoscope of claim 1, wherein said interface is formed in such a way that light can be transmitted from said operating part to said working part.

4. The video endoscope of claim 1, wherein said working part comprises an outer part formed as a shaft.

5. The video endoscope of claim 1, further comprising at least one light guide, said at least one light guide being arranged in said shaft.

6. The video endoscope of claim 1, wherein said working part comprises an inner part, in which said image sensor and said electrical transmission system are arranged.

7. The video endoscope of claim 4, wherein said working part comprises an inner part, in which said image sensor and said electrical transmission system are arranged.

8. The video endoscope of claim 7, wherein said outer part can be rotated relative to said inner part.

9. The video endoscope of claim 4, wherein a mechanism for rotating said outer part is provided.

10. The video endoscope of claim 4, wherein said image sensor is formed as a CMOS sensor.

11. The video endoscope of claim 1, wherein said image sensor is formed as a CCD sensor.

12. The video endoscope of claim 1, wherein said electrical transmission system is formed as at least one continuous circuit board having a distal end and a proximal end.

13. The video endoscope of claim 12, wherein said proximal end of said circuit board is formed as a plug.

14. The video endoscope of claim 12, wherein said image sensor and said circuit board are enclosed by a cylindrical element.

15. The video endoscope of claim 1, wherein said working part comprises at least one channel.

16. The video endoscope of claim 1, wherein said proximal end of said working part has a larger diameter than said distal end of said working part.

17. The video endoscope of claim 1, further comprising LEDs, said LEDs being arranged in said working part.

18. The video endoscope of claim 6, further comprising LEDs, said LEDs being arranged in said working part.

19. The video endoscope of claim 18, wherein said LEDs are arranged in said inner part.

20. The video endoscope of claim 1, further comprising a light entrance, said light entrance being arranged at said proximal end of said working part.

21. The video endoscope of claim 1, further comprising a light exit, said light exit being arranged at said distal end of said operating part.

22. The video endoscope of claim 13, further comprising a socket for receiving said plug, said socket being arranged at said distal end of said operating part.

23. The video endoscope of claim 1, wherein said operating part comprises operating elements.

24. The video endoscope of claim 20, further comprising LEDs, said LEDs being arranged circumferentially in the region of said distal end of said operating part, said LEDs illuminating said light entrance of said working part.

25. The video endoscope of claim 20, further comprising optical fibres, said optical fibres being arranged in a manner distributed circumferentially in the region of said distal end of said operating part, said optical fibres illuminating said light entrance of said working part.

26. The video endoscope of claim 20, further comprising LEDs, said LEDs being arranged in a manner distributed circumferentially in said operating part, said LEDs coupling light radially into said working part, which can be deflected towards said distal end of said working part by means of mirror elements.
